(19) United States
(12) Patent Application Publication
(10) Pub. No.: US 2016/0015890 A1
(43) Pub. Date: Jan. 21, 2016

(54) SYSTEM AND METHOD OF VARIABLE DOSE GLUCAGON DELIVERY

(71) Applicant: Artificial Pancreas Technologies, Inc., Santa Barbara, CA (US)

(72) Inventors: Eyal Dassau, Goleta, CA (US); Howard Craig Zisser, Santa Barbara, CA (US); Samir Suresh Mitragotri, Santa Barbara, CA (US); Francis J. Doyle, III, Santa Barbara, CA (US)

(21) Appl. No.: 14/734,994

(22) Filed: Jun. 9, 2015

Related U.S. Application Data
(60) Provisional application No. 61/997,792, filed on Jun. 9, 2014.

(51) Int. Cl. A61M 5/14; A61M 5/168

(52) U.S. Cl. CPC A61M 5/1409; A61M 5/16827; A61M 2202/07

(57) ABSTRACT

Disclosed are systems and methods for variable dose glucagon delivery without the need to manually reconstitute the glucagon. These systems and methods mat be utilized with or without a separate continuous glucose monitor. The variability of the dose may be determined manually by the user, caregiver or responder, or by preprogrammed algorithms utilizing the input from an external sensor such as a continuous glucose monitor.

### CROSS REFERENCE TO RELATED APPLICATIONS

This application is based upon and claims the benefit of U.. provisional application No. 61/997,792, entitled “System and Method of Variable Dose Glucagon Delivery”, filed Jun. 9, 2014, the entire disclosure of which is hereby specifically incorporated by reference for all that it discloses and teaches.

### BACKGROUND OF THE INVENTION

Carbohydrate metabolism disorders and blood sugar regulation disorders such as diabetes, especially when treated with intensive insulin therapy, have inherent risk of hypoglycemia that can lead to loss of consciousness, cardiac arrhythmia, seizure, and death. Whereas glucagon can be injected subcutaneously to reverse the hypoglycemia effects of insulin, it is currently only available in dry form, as no soluble agent has been developed that has proven to be safe and effective.

Glucagon is currently being used for diabetes in one of two ways: (1) as part of a rescue kit, typically in the form of a pen-like device, which is used either by the person with diabetes, or by a caregiver or emergency responder, to rescue the person with diabetes from a severe hypoglycemic event; or, (2) as part of a bi-hormonal system that monitors glucose levels and delivers either insulin or glucagon. In either case, the need for soluble glucagon near the time it is to be injected, limits the utility of the glucagon. In the case of the rescue kit, the predetermined dose may or may not be appropriate for the degree of hypoglycemia. In the case of the bi-hormonal system, the glucagon is only available to persons willing to subject themselves to the closed loop control of the bi-hormonal system, including automated insulin delivery, and soluble glucagon, which must be made available much more frequently than the insulin needs to be replenished, making it only suitable for use in a clinical setting.

### SUMMARY OF THE INVENTION

An embodiment of the present invention may therefore comprise: a system for variable dose glucagon delivery to a patient without the need to manually reconstitute the glucagon comprising: a reconstitution mechanism comprising: a reconstitution chamber disposed between an inlet and an outlet; a stabilized solute form of glucagon; a solute delivery mechanism for metered dosing of the solute glucagon into the reconstitution chamber; a diluent supply in fluid communication with the reconstitution chamber; an activation switch that triggers the solute delivery mechanism to provide a metered dose of the solute glucagon into the reconstitution chamber, and triggers the diluent supply to provide a metered dose of a diluent into the reconstitution chamber; and, a delivery mechanism disposed in fluid communication with the outlet to controllably provide a predetermined amount of a soluble glucagon solution to a tissue of the patient.

An embodiment of the present invention may also comprise: a method for providing a variable dose of glucagon delivered to a patient without the need to manually reconstitute the glucagon comprising: metering and dispensing a dose of stabilized solute form of glucagon to a reconstitution chamber; metering and dispensing a diluent dose to the reconstitution chamber to create a soluble glucagon solution of predetermined concentration; triggering a release of the glucagon solution from the reconstitution chamber based upon a physiological condition of the patient; and, controllably delivering the released glucagon solution to provide a predetermined amount of the soluble glucagon solution to a tissue of the patient.

### BRIEF DESCRIPTION OF THE DRAWINGS

In the drawings,

FIG. 1 illustrates an embodiment of a variable dose glucagon delivery system in which the reconstitution mechanism is in the form of a mechanical pencil.

FIG. 2 illustrates another embodiment of a variable dose glucagon delivery system in which the reconstitution mechanism utilizes a glucagon mesh.

FIG. 3 illustrates another embodiment of a variable dose glucagon delivery system in which the reconstitution mechanism utilizes a mechanical extruder.

FIG. 4 illustrates another embodiment of a variable dose glucagon delivery system in which insulin and glucagon are administered through different portals.

### DETAILED DESCRIPTION OF THE INVENTION

While this invention is susceptible to embodiment in many different forms, it is shown in the drawings, and will be described herein in detail, specific embodiments thereof with the understanding that the present disclosure is to be considered as an exemplification of the principles of the invention and is not to be limited to the specific embodiments described.

As stated above, diabetes, especially when treated with intensive insulin therapy, has an inherent risk of hypoglycemia that can lead to loss of consciousness, cardiac arrhythmia, seizure, and death. Glucagon can be injected subcutaneously to reverse the hypoglycemia effects of insulin. Glucagon is currently only available in dry form, as no soluble glucagon has been proven safe and effective.

The disclosed system overcomes the current limitations by automatically reconstituting glucagon in the desired dose, either as a dose that is manually selected, or as determined using the input of a separate continuous glucose monitor connected to the system wirelessly or by wire. The disclosed system can be worn, can be used as a rescue kit, can be utilized as part of a bi-hormonal system, or in new applications enabled by the system, such as basal dosing or prevention of nighttime hypoglycemia.

The need to manually reconstitute glucagon at the approximate time it is to be injected, ultimately limits the utility of the glucagon. The disclosed system delivers the desired dose automatically, without the need to manually reconstitute the glucagon. This enables variable dosing for rescue purposes that better corresponds with the degree of hypoglycemia, and in bi-hormonal systems, eliminating the need to manually reconstitute the glucagon.

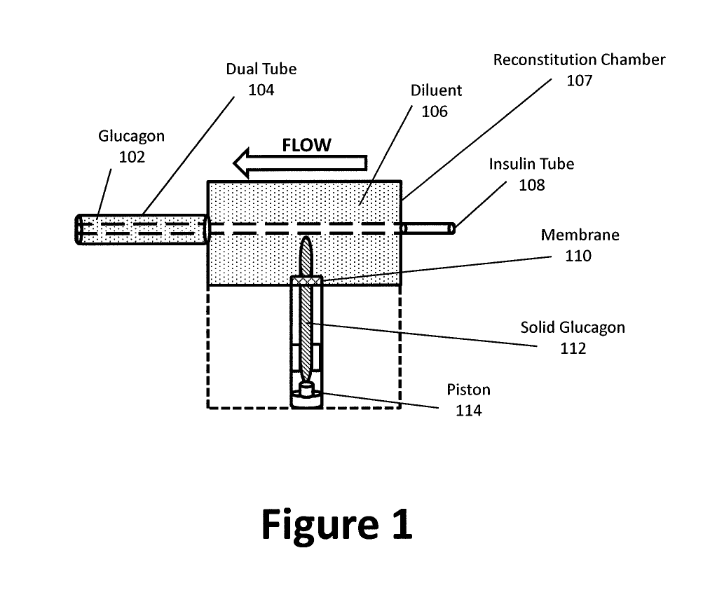

Thus, the disclosed embodiments include a reconstitution mechanism and a delivery mechanism. The reconstitution mechanism may be of various forms including, but not limited to the disclosed embodiments. FIG. 1 illustrates an embodiment of a variable dose glucagon delivery system in which the reconstitution mechanism is in the foam of a mechanical pencil. This system involves utilization of solid or semi-solid forms of glucagon. Conventional, commercially available glucagon typically possesses poor solubility in aqueous buffers at or near physiological pH values. At higher and lower pH values, at which the peptide can be formulated to concentrations of a milligram or more per milliliter, the chemical integrity of the hormone can be limited, which is evidenced by the formation of multiple degradation-related peptides.

Consequently, commercial preparations are typically provided, for example, as a lyophilized solid with an acidic diluent for rendering it soluble at the time for immediate use. Any unused material must be disposed of immediately after initial use due to degradation, thereby further enhancing the need for a system that can utilize a metered approach to reconstitution.

In the embodiment disclosed in FIG. 1, semi-solid or solid glucagon 114 processed in the form of a cylinder or rod for instance, may be inserted manually or pushed by a drive mechanism such as a piston 114 into a reconstitution chamber 107 where it is reconstituted with a diluent 106 that is in communication with a conduit that infuses the liquid from the device into a patient's body. In this embodiment, the conduit is a dual tube 104 that mixes influent from the insulin tube 108 with the reconstituted glucagon 102 and allows a precise metered amount of solid glucagon 102 to be reconstituted and introduced to the patient in a form that may be analogous to a mechanical pencil.

In this form, each ‘click’ of the device pushes a pre-defined dose of solid glucagon through a membrane 110 into the reconstitution chamber 107 that continuously infuses saline or other diluent 106 for reconstitution. The number of ‘clicks’ can be adjusted to match the necessary dose of glucagon 102 based upon the instantaneous physiological need of the recipient. It is also contemplated within the disclosure, that a variety of injection mechanisms, such as an indexed screw drive or other types, may also be utilized to deliver a specific amount of solid glucagon 112 into the reconstitution chamber.

A unique advantage of this approach is that solid glucagon 112 can be pushed into saline without the use of additional devices, since the glucagon rod may perform the task of piercing the confines of the reconstitution chamber in a membrane or the tubing. Once inserted, glucagon will readily dissolve, thus formulating the dose within the liquid prior to infusion to the recipient.

The tubing and/or membrane 110 may be formulated with a flexible polymer such that the glucagon rod can be pushed into the reconstitution chamber or tubing without leaking the saline. Utilizing this approach, solid glucagon is processed into long, rod-shaped objects. The rods are able to maintain sufficient mechanical integrity to pierce the chamber membrane 110 or tubing wall. This may be facilitated by using inert additives incorporated with or within the solid glucagon mixture, for example, salts or the like, which enable the enhanced mechanical composition while remaining biologically inert. To prepare such a composition, lyophilized glucagon, for example, may be mixed with appropriate salts or other inert agents (e.g. sodium chloride, calcium chloride, potassium chloride, or the like) and compressed into an appropriate shape, such as a cylinder or rod.

The solid glucagon 112 composition provides unique structural, chemical and biological properties providing high biological activity and selectivity, while additionally possessing sufficient aqueous solubility and stability to be utilized as a ready-to-use pharmaceutical agent. The above embodiments allow for glucagon delivery (other than zero if delivery is suspended) in the resolution range of approximately 5 micro-grams. An upper end delivery of 1 mg of glucagon diluted in 1 ml of diluent may be delivered over a period of 10 minutes.

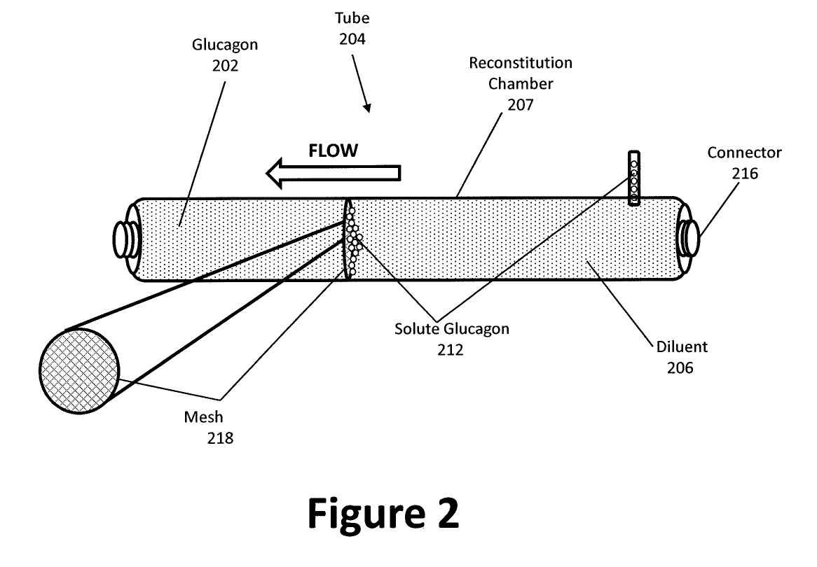

FIG. 2 illustrates another embodiment of a variable dose glucagon delivery system in which the reconstitution mechanism utilizes a glucagon mesh. In this embodiment, solute glucagon is deposited on, or introduced upstream, of an insoluble mesh 218 (sieve/filter) disposed within a conduit, such as a tube 204. The tube unit may be placed in an infusion line and act as both a reconstitution mechanism and a delivery mechanism as the tube 204 acts to facilitate the flow of diluent 206 (e.g., saline) through the mesh and dissolve the requisite dose of solute glucagon 212 within the reconstitution chamber 207. The mesh size or sieving coefficient of the mesh 218, and the amount of glucagon deposited on, or upstream of the sieve, allows the solute glucagon 212 to sufficiently dissolve before infusion as glucagon 202, while the porosity facilitates flow of the diluent 206 without significant pressure drop. The tube 204 may contain connectors to easily facilitate in-line connection and communication.

In this embodiment, the tube 204 may be pre-packaged to deliver a particular dose of glucagon, which may be of a variety of standard doses or the tubes 204 may be placed in series to obtain higher doses. The tube 204 may also have a mechanism of delivering solute glucagon 210 into the reconstitution chamber 207 upstream of the mesh 218.

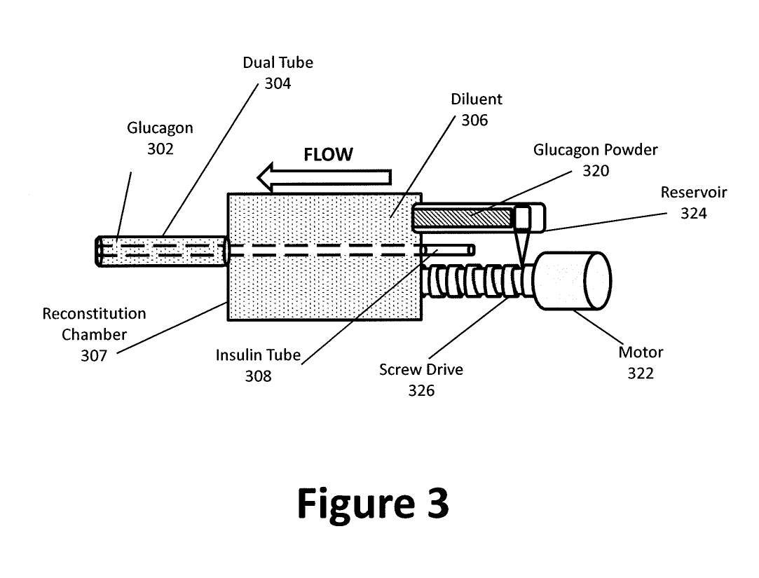

FIG. 3 illustrates another embodiment of a variable dose glucagon delivery system in which the reconstitution mechanism utilizes a mechanical extruder type infusion. Utilizing this embodiment facilitates delivery of solid amorphous glucagon powder 320 from a reservoir 324 into a reconstitution chamber 307 with diluent 306 (e.g., saline) flowing from an insulin tube 308 to a patient via a dual tube 304. The extruder type mechanism may comprise a screw drive 326 with pre-determined pitch, driven with a rotational motor 322 with precise speed, or a manual indexing knob so as to control precise delivery of the solid powder. This embodiment offers an advantage of translocating solids, without blockage, while maintaining required mass delivery rate into the diluent.

The aforementioned reconstitution embodiments may be used with a variety of delivery mechanisms such as a patch system, an insulin tubing extension or as a multi-chamber pump.

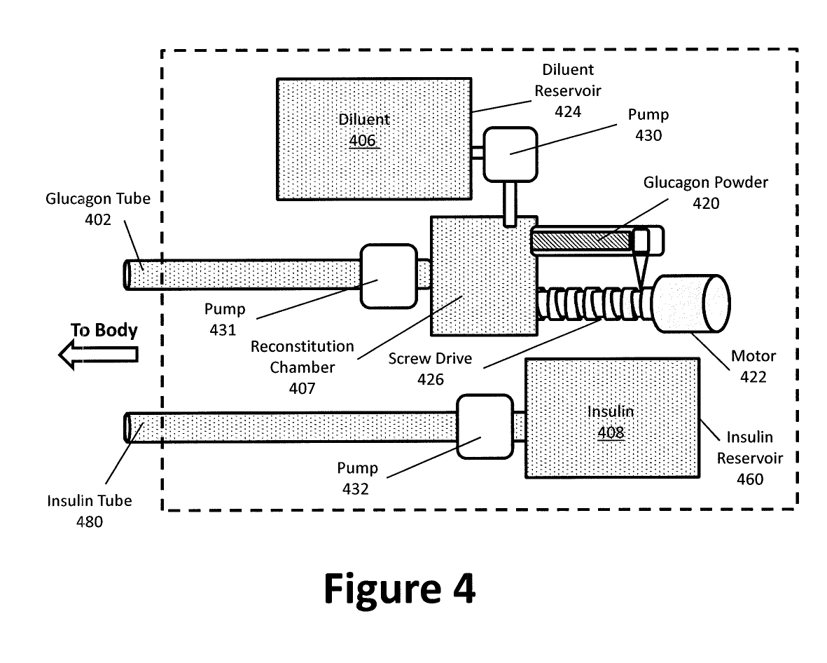

FIG. 4 illustrates another embodiment of a variable dose glucagon delivery system in which the reconstitution mechanism utilizes a mechanical extruder type infusion, and the biomolecules (insulin and glucagon) are administered into the body through separate and distinct portals. Utilizing this embodiment facilitates delivery of solid amorphous glucagon powder 420 into a reconstitution chamber 407 with diluent 406 (e.g., saline) delivered via a pump 430 from a diluent reservoir 424. The extruder type mechanism may comprise a screw drive 426 with pre-determined pitch, driven with a rotational motor 422 with precise speed or a manual indexing knob, so as to control precise delivery of the solid powder. This embodiment offers an advantage of translocating solids without blockage while maintaining required mass delivery rate into the diluent. The resultant glucagon solution is delivered into the body using a glucagon tube 402 and optionally a glucagon pump 431. The device also comprises an insulin reservoir 460 whose rate of administration of insulin 408 is controlled via a pump 432 and the delivery into the body is mediated through an insulin tube 480.

The aforementioned reconstitution embodiments may be used with a variety of delivery mechanisms such as a patch system, an insulin tubing extension or as a multi-chamber pump.

Utilizing a patch system allows the system to be worn on a patient's skin. The device is adhered to the skin of the user using appropriate adhesives, which may include, but are not limited to; acrylic, polyisobutylene, silicone, hybrid chemistries or the like, that are tailored to bond in various environments for wear times that may range from minutes to weeks.

The insulin tubing extension embodiment is a continuous infusion device. Continuous infusion ensures that the tubes are free of blockage and the microenvironment in the subcutaneous space at the injection site is maintained. The infused diluent (saline or the like) may contain insulin, glucagon or both. Insulin may be added to the infused diluent by providing a controlled addition from the reservoir and glucagon may be added using any of the delivery mechanisms described herein to provide continuous infusion.

The multi-chamber pump embodiment may include two or more chambers, and is typically a two chamber system with one chamber for insulin and one for glucagon. Glucagon may be stored in the device in a solid (or semi-solid) form and delivered into the body after reconstitution as described herein.

The systems described herein can be used in a rescue mode and/or non-rescue mode. In the rescue mode, the patient/caregiver/first responder pushes a button to activate the pump and/or the reconstitution mechanism to rapidly dispatch a “rescue” dose. The purpose of this rescue dose is to avoid severe hypoglycemia. The rescue dose may typically range from 0.5 to 1.0 mg to be delivered over a period of time, for example, ten minutes. This rescue dose is approximately within the 0.02-0.03 mg/kg of body weight range in the pediatric population, and approximately 1.0 mg/kg of body weight range in adults.

The rescue dose may also be activated based upon automated or real time glucose measurements. In this mode, a signal from a continuous glucose monitor is utilized in determining the activation of the rescue dose. Glucagon release may be triggered, for instance, if the glucose concentration is lower than a predefined low level (e.g. <50 mg/dL for more than 20 minutes), or the glucose rate of change is negative and the glucose level is low (e.g. <60 mg/dL with a rate of change (ROC) of −1 mg/dL/min or lower). This mode may be defined as a threshold activation, which can release a full rescue dose.

In the continuous delivery mode, glucagon is used to offset the effects of excessive insulin delivery. In this mode, a micro-dose of glucagon may be released based upon a glucose ROC estimation, or other model, that can calculate a required dose capable of compensating for the excessive amount of insulin. A simple control algorithm, such as a proportional-integral-derivative (PID), or a more sophisticated one, such as Model Predictive Control (MPC), can be used to estimate the required action.

In both rescue mode, as well as continuous mode, glucagon may be delivered in the following manner. The controller (or the operator) determines a requisite dose of glucagon to be delivered. This requisite dose is dispensed from the reservoir into the delivery tubing utilizing for example, one or more of the embodiments described herein. Saline/insulin is distributed through the tubing until an affecting therapeutic dose of glucagon is delivered to the user.

With the exception of the triggering algorithms, the individual system components may be based upon pre-existing components, which may be assembled as described with minimal modification. The activation may be embedded into the system as a separate continuous glucose monitor, or in a third party or external device such as a smartphone. Ideally these systems may be integrated into a closed loop artificial pancreas.

As embodied herein, a patient with a carbohydrate metabolism disorder, such as diabetes, may wear the system, with or without a connected (wirelessly or by wire) continuous glucose monitor. The disclosed systems may be operated automatically or manually, and can be utilized as a standalone device or integrated into a larger system such as a closed-loop artificial pancreas.

The foregoing description of the invention has been presented for purposes of illustration and description. It is not intended to be exhaustive or to limit the invention to the precise form disclosed, and other modifications and variations may be possible in light of the above teachings. The embodiment was chosen and described in order to best explain the principles of the invention and its practical application to thereby enable others skilled in the art to best utilize the invention in various embodiments and various modifications as are suited to the particular use contemplated. It is intended that the appended claims be construed to include other alternative embodiments of the invention except insofar as limited by the prior art.

The embodiments of the invention in which an exclusive property or privilege is claimed are defined as follows:

1. A system for variable dose glucagon delivery to a patient without the need to manually reconstitute the glucagon comprising:
a reconstitution mechanism comprising:
a reconstitution chamber disposed between an inlet and an outlet;
a stabilized solute form of glucagon;
a solute delivery mechanism for metered dosing of said solute glucagon into said reconstitution chamber;
a diluent supply in fluid communication with said reconstitution chamber;
an activation switch that triggers said solute delivery mechanism to provide a metered dose of said solute glucagon into said reconstitution chamber, and triggers said diluent supply to provide a metered dose of a diluent into said reconstitution chamber; and,
a delivery mechanism disposed in fluid communication with said outlet to controllably provide a predetermined amount of a soluble glucagon solution to a tissue of said patient.

2. The system of claim 1, wherein said glucagon is in a solid or semi-solid form.

3. The system of claim 1, wherein said reconstitution mechanism further comprises, said solute glucagon that is deposited on, or introduced upstream, of an insoluble sieve disposed within said reconstitution chamber.

4. The system of claim 3, wherein said insoluble sieve has a mesh size or sieving coefficient sufficient to allow said solute glucagon to sufficiently dissolve before exiting said reconstitution chamber.

5. The system of claim 1, wherein said solute delivery mechanism further comprises:
a motorized mechanism that meters and dispenses precise amounts of said solute glucagon from a reservoir to said reconstitution chamber.

6. The system of claim 1, wherein said solute glucagon further comprises; lyophilized glucagon.

7. The system of claim 1, wherein said solute glucagon further comprises; lyophilized glucagon mixed with inert binding agents.

8. The system of claim 1, wherein said activation switch relies upon an input from a human to trigger dosing of said glucagon to said patient.

9. The system of claim 1, wherein said activation switch relies upon a measurement of a physiological condition of said patient to trigger dosing of said glucagon to said patient.

10. The system of claim 1, further comprising:
an insulin reservoir containing insulin; and,
an insulin pump that pumps insulin through an insulin tube for delivery into said patient.

11. The system of claim 10, wherein said activation switch relies upon a measurement of a physiological condition of said patient to trigger dosing of said glucagon and said insulin to said patient.

12. The system of claim 1, further comprising:
a diluent reservoir containing a supply of said diluent; and,
a diluent pump that pumps said diluent into said reconstitution chamber to supply a glucagon solution through a glucagon tube for delivery into said patient.

13. A method for providing a variable dose of glucagon that is delivered to a patient without the need to manually reconstitute the glucagon comprising:
metering and dispensing a dose of stabilized solute form of glucagon to a reconstitution chamber;
metering and dispensing a diluent dose to said reconstitution chamber to create a soluble glucagon solution of predetermined concentration;
triggering a release of said glucagon solution from said reconstitution chamber based upon a physiological condition of said patient; and,
controllably delivering said released glucagon solution to provide a predetermined amount of said soluble glucagon solution to a tissue of said patient.

14. The method of claim 13, further comprising the step:
metering and dispensing said dose of stabilized solute form of glucagon to said reconstitution chamber in a solid or semi-solid form.

15. The method of claim 13, further comprising the step:
depositing said solute glucagon upon, or introducing said solute glucagon upstream, of an insoluble sieve disposed within said reconstitution chamber.

16. The method of claim 15, further comprising the step:
entraining said solute glucagon with said insoluble sieve within said reconstitution chamber thereby allowing said solute glucagon to sufficiently dissolve before exiting said reconstitution chamber.

17. The method of claim 13, further comprising the step:
metering and dispensing a precise amount of said solute glucagon from a reservoir to said reconstitution chamber with a motorized mechanism.

18. The method of claim 13, wherein said step of metering and dispensing a dose of stabilized solute form of glucagon, further comprises:
metering and dispensing said dose comprising lyophilized glucagon.

19. The method of claim 13, wherein said step of metering and dispensing a dose of stabilized solute form of glucagon, further comprises:
metering and dispensing said dose comprising lyophilized glucagon mixed with inert binding agents.

20. The method of claim 13, further comprising the step:
manually triggering a release of said glucagon solution from said reconstitution chamber based upon input from a human.

21. The method of claim 13, further comprising the step:
measuring a physiological condition of said patient and utilizing the result of said measurement for said triggering and said delivery of said glucagon to said patient.

22. The method of claim 13, further comprising the step:
metering and dispensing a precise amount of insulin from a an insulin reservoir through an insulin tube for delivery into said patient.

23. The method of claim 22, further comprising the step:
triggering said metering and dispensing of said glucagon and said insulin to said patient based upon a measurement of a physiological condition of said patient.

24. The method of claim 13, further comprising the step:
providing a diluent reservoir containing said diluent; and,
pumping said diluent into said reconstitution chamber to supply a glucagon solution through a glucagon tube for delivery into said patient.
